(19) United States
(12) Patent Application Publication
(10) Pub. No.: US 2010/0298740 A1
(43) Pub. Date: Nov. 25, 2010

(54) Diagnostic system for accurate recording of acoustic signals

(75) Inventors: Gregory Gelman, Rehovot (IL); Igal Kushnir, Pardes Hana (IL); Yael Glickman, Haifa (IL)

Correspondence Address: Ronny Fogel, 8 Gordon, Givatayim, 53235, IL

(73) Assignee: Deep Breeze Ltd., Or Akiva (IL)

(21) Appl. No.: 12/801,719

(22) Filed: Jun. 22, 2010

(30) Foreign Application Priority Data
Dec. 30, 2007 (IL) PCT/IL2007/001622

(51) Int. Cl. A61B 7/00

(52) U.S. Cl. 600/586

(57) ABSTRACT

The present invention discloses a novel positioning unit configured in accordance with a recording matrix for ensuring correct and ergonomic positioning of a patient with respect to the recording matrix utilizing body landmarks such as spine and scapula. The technique of the present invention provides for supporting at least a region of the patient body in a desired position (e.g. on a patient bed) and positioning the regions of the patient body relative to the patient bed. The positioning unit allows repeated, precise supporting and positioning of a patient in a desired position with the patient being comfortably supported or fixed in this position, during the monitoring and diagnostic procedure, i.e. when the recording matrix is applied to the patient.

### FIELD OF THE INVENTION

The present invention relates to diagnostic systems and techniques based upon the review and analysis of acoustic signals coming from a body, and more particularly to a diagnostic system for accurate recording of such signals.

### BACKGROUND OF THE INVENTION

Medical devices are often applied to a subject's body in order to measure or record a parameter of the body or to apply a stimulus thereto. Such devices include, for example, thermometers, stethoscopes, electrodes, microphones, and so on. If the device is to be applied to the body for a short period of time (e.g. a stethoscope), it may be applied manually by a care giver. If the device is to be applied to the body for a prolonged period of time (e.g. ECG electrodes), the device is usually affixed to the body. Such means of fixation include, for example, use of straps, embedding the device in a garment worn by the subject, or by means of vacuum.

Body sounds (or generally “acoustic signals”) are routinely used by physicians in the diagnosis of various disorders. A physician may place a stethoscope on a person's chest or back and monitor the person's breathing or heart sounds in order to detect adventitious (i.e. abnormal or unexpected) body sounds.

The identification and classification of these adventitious sounds often provide important information about physiological abnormalities. For example, U.S. Pat. No. 5,010,889, entitled intelligent stethoscope to Bredesen et al., discloses a stethoscope capable of digitizing and storing body sounds, including heart and lung sounds, in a memory structure configured to store up to six different sounds. The stethoscope includes a single chest piece with a microphone, which may be moved to one of six locations around the patient's chest. Using waveform signature analysis, each of the six recorded waveforms is examined to determine the presence of high-pitch sounds which may correspond to fine crackles or low-pitch sounds which may correspond to coarse crackles. The presence or absence of these sounds is then formed into an array that may be compared with pre-recorded arrays corresponding to known conditions, e.g., normal lung sounds, pneumonia, etc. If a match is found between the recorded waveforms and one of the pre-recorded arrays, a diagnosis may be displayed on the LCD panel of the stethoscope.

A microphone can be affixed to the body in order to record body sounds. The recorded sound signals may be amplified and filtered before being listened by a physician. The recorded signals may also be analyzed by signal processing techniques. Moreover, a plurality of microphones can be affixed over a body surface in order to obtain a plurality of sound signals simultaneously from the body surface. For example, U.S. Pat. No. 6,394,967 discloses a system in which a plurality of microphones is affixed to a person's back or chest for recording respiratory tract sound. The microphones are affixed to the body surface using tape or straps to prevent dislocation or movement during the data acquisition process. The method of attachment by adhesive tape is simple but does not provide for the easy displacement of the device to another site of the skin, in case it had not been put in the right place from the beginning. Moreover, adhesive tape may cause inconvenience when removed from areas with hair growth, may fall off through the effect of transpiration, or may, in the case of incautious handling during application, fasten in places not considered for application. With said fixation arrangements, it is moreover not easy to bring into skin contact diagnostic and therapeutic devices in a way that guarantees a constant force over time to be exerted by the device onto the skin. Keeping the force of attachment constant is important in receiving electrical or acoustic signals through the skin.

Another technique for affixing a microphone to a body surface is by means of suction. For example, U.S. Pat. No. 4,777,961 discloses a microphone embedded in the wall of a dome shaped stethoscope head formed from an elastic flexible material. The stethoscope head is squeezed by fingertip pressure as it is applied to the body surface. When the fingertip pressure is removed, the head expands slightly so as to create a partial vacuum in its interior so as to keep the head affixed to the body surface. The membrane of the microphone, however, is not pressed against the body surface.

Another technique for affixing a microphone to a body surface is by vacuum. For example, U.S. Pat. No. 4,736,749 discloses a holder for a signal pick-up device, such as a microphone or electrode that is fixed to the body surface by vacuum. The holder has a chamber that is evacuated by an external source of negative pressure. When the chamber is evacuated, the holder is held firmly to the skin and the signal-pick up device is pressed to the skin inside the chamber.

U.S. Pat. No. 6,887,208, assigned to the assignee of the present application, discloses a system in which a plurality of microphones are affixed to a person's back or chest for recording respiratory tract sounds. This patent teaches embedding the microphones in a matrix that may be in the form of a vest or garment securely worn by the person during signal acquisition. Different sized or shaped matrices may be used for differently sized individuals, for different sexes, ages, etc.

However, in many cases, functioning of a device applied to a subject's body requires firm contact between the device and the body. In practice, the presence of skin features, such as hair, pimples or wrinkles, prevent firm application of the device to the skin. The problem is especially significant when a device is to be applied to the skin by vacuum. The presence of skin features often prevents the formation of an airtight seal between the device and the skin.

### GENERAL DESCRIPTION

There is a need in the art in reproducibly and accurately positioning and supporting a patient during a medical procedure (diagnosis and/or treatment). The present invention solves this problem by providing a novel positioning unit configured in accordance with a recording matrix for ensuring correct and ergonomic positioning of a patient with respect to the recording matrix utilizing body landmarks such as spine and scapula. The technique of the present invention provides for supporting at least a region of the patient body in a desired position (e.g. on a patient bed) and positioning the regions of the patient body relative to the patient bed. The positioning unit allows repeated, precise supporting and positioning of a patient in a desired position with the patient being comfortably supported or fixed in this position, during the monitoring and diagnostic procedure, i.e. when the recording matrix is applied to the patient.

In some embodiments, the recording matrix is designed for the evaluation of respiratory function, the monitoring of patients with lung conditions and their response to treatment. The recording matrix may also be intended to use for cardiac examination.

Using a non-invasive, radiation-free technique, the recording matrix utilizes vibration response imaging technology to monitor the breath sounds produced by the lungs during the breathing cycle. The data acquisition is achieved by using an array of pressure sensors (e.g., microphone matrix) placed in contact with the patient's back.

It should be noted that the term “microphone” used herein refers to any suitable known type of a pressure sensors.

It should be noted that the diagnostic system, i.e. the positioning unit and the recording matrix attached thereto, is configured to provide both ergonomic benefit and improved diagnostic accuracy through improved access to pertinent anatomy. For example, the positioning unit may be reconfigured to back repositioning of obese individuals who otherwise present back configurations that present ergonomic difficulties to the image taking technician or awkwardness to the patient for back self-positioning. The positioning unit may also be configured for the ergonomic immobilization of other organs and body regions to better obtain an image of clinical interest.

Moreover, since the patient is positioned on the patient bed by the positioning unit, the positioning unit may be disposable, intended for single-patient use, reducing the possibility of cross-contamination.

According to one broad aspect of the present invention, there is provided a diagnostic system including at least one matrix carrying an array of sensors for recording signals from a body, and a positioning unit configured for alignment with the matrix. The positioning unit has a surface relief matching a surface relief of at least a part of the matrix, such that the positioning unit is adapted for engaging with at least part of the array of sensors matrix and maintaining the matrix when in operation, thereby enabling accurate positioning of the sensors with respect to a body region from which the signals are to be recorded. The positioning unit includes a frame made with at least one opening of a geometry defining the surface relief for accommodating at least one array of sensors. The opening has a geometry matching the geometry of at least a region of the matrix. The opening defines an array of notches matching the shaped contour of the matrix-column to receive the sensors of the column within the notches respectively.

In some embodiments, the opening defines an array of N notches wherein N is superior to the number of the columns of the array of sensors matrix. The diagnostic system is configured to provide a plurality of height and width adjustable positions.

The positioning unit may be disposable.

The diagnostic system is configured and operable to ensure correct and ergonomic positioning.

In some embodiments, the diagnostic includes two matrixes; the positioning unit having a frame made with two openings configured to accommodate the two matrices enabling the simultaneous recording of the acoustic signals of the right and the left side of a patient. The positioning unit may include a disposable interface covering at least the entire upper surface thereof. A spine pointer may be marked on the positioning unit such that the patient's spine is properly aligned relatively to the matrix.

The matrix is configured to be flexible and conform to the shape of a surface applied to it.

In some embodiments, the diagnostic system is configured such that the positioning unit and/or the recording matrix is/are configured as an inflating structure enabling the contact of all the sensors of the matrix with the patient's body.

According to another broad aspect of the present invention, there is provided a positioning unit for use in a diagnostic system; the positioning unit being configured to define a surface relief matching a surface relief of at least a part of a multiple-sensor matrix, such that the positioning unit is adapted for engaging with the at least part of the matrix and maintaining the matrix when in operation.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 illustrates an exemplary configuration of the diagnostic system comprising a positioning unit and two microphones matrices;

FIG. 2 represents an exemplary configuration of the positioning unit of the present invention;

FIG. 3 illustrates the positioning of the diagnostic system of the present invention respectively to the subject to be examined;

FIG. 4 illustrates a pair of matrix having ergonomic capsules according to one embodiment of the present invention;

FIG. 5 illustrates another exemplary configuration of the positioning unit of the present invention;

FIG. 6 illustrates the alignment of a left microphone array on the notched surface relief of the positioning unit on the side labeled L;

FIG. 7 illustrates the alignment of the two microphone arrays on the notched surface relieves of the positioning unit on the sides labeled L and R;

FIG. 8 illustrates the covering of the microphone arrays by folding over a sheath attached to the positioning unit;

FIG. 9 illustrates the positioning unit in a folded shape;

FIG. 10 illustrates the diagnostic system configured as an inflating structure; and;

FIG. 11 illustrates the positioning unit configured as an inflating structure.

### DETAILED DESCRIPTION OF EXEMPLARY EMBODIMENTS

The present invention utilizes a recording matrix configured as a plurality of pressure sensors (e.g. microphones) arranged in a one- or two-dimensional array, and then kept together within a single structure/housing. The present invention is mainly intended for use with a pressure sensors' array for recording signals from a body being monitored and/or treated and/or diagnosed. The sensors' array is capable of detecting the vibration response from the body caused by air movement through the lungs during the respiratory cycle.

When a body surface, such as a person's back is applied onto the matrix, the microphones in the matrix are pressed by the body to create sufficient acoustic coupling between the body surface and the microphones. Thus, with the back of the individual properly positioned on the upper surface of the microphone matrix, at least some of the weight of the individual is transferred to the upper surface of the matrix. In this way, the microphones are firmly applied to the individual's back without the need of an attachment system, such as straps, tape, or vacuum. Respiratory tract sounds, or other acoustic signals originating in the thorax are detected by the microphones in the matrix.

It should be noted that the housing of the microphone matrix acoustically isolates the sensors from the surrounding. The housing (preferably made of silicon) surrounds the sensor as a sound insulation, which is intended to attenuate environmental noise. Therefore, the sensor is not in direct contact neither with the air surrounding, nor with the mattress, resulting in isolation from extraneous noise.

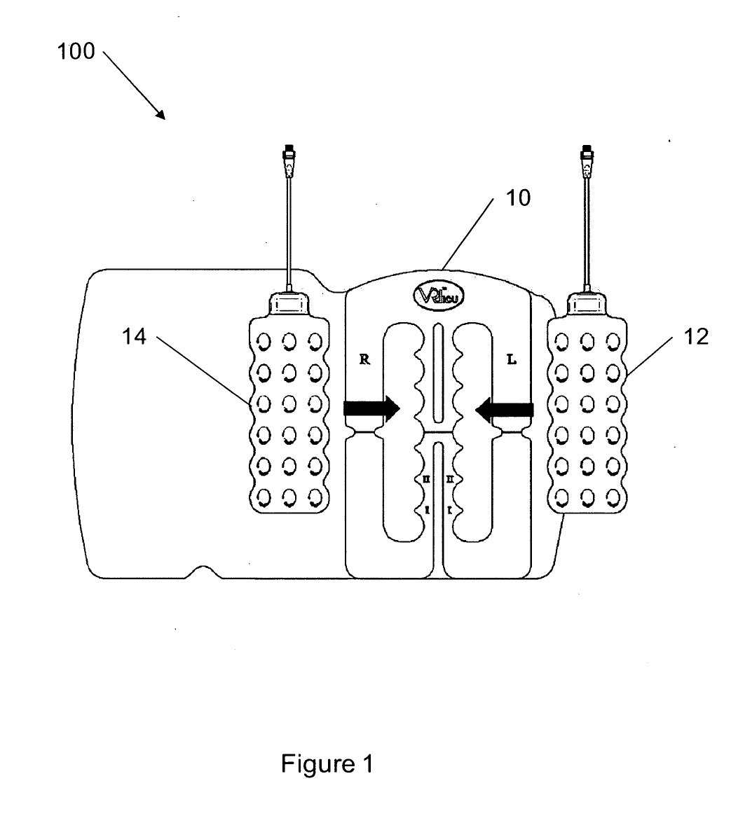

Reference is made to FIG. 1 illustrating a diagnostic system 100 in accordance with one embodiment of the invention. The system 100 includes a positioning unit 10 configured to be attached to a pair of matrices, left 12 and right 14, being two matrices to be aligned on the positioning unit for recording acoustic signals. In this non-limiting example, each matrix comprises an array of pressure sensors (microphones) encased in pads of medical-grade silicone and arranged in equally-spaced rows and columns. To facilitate understanding, the same reference numbers will be used for identifying components that are common in all the examples.

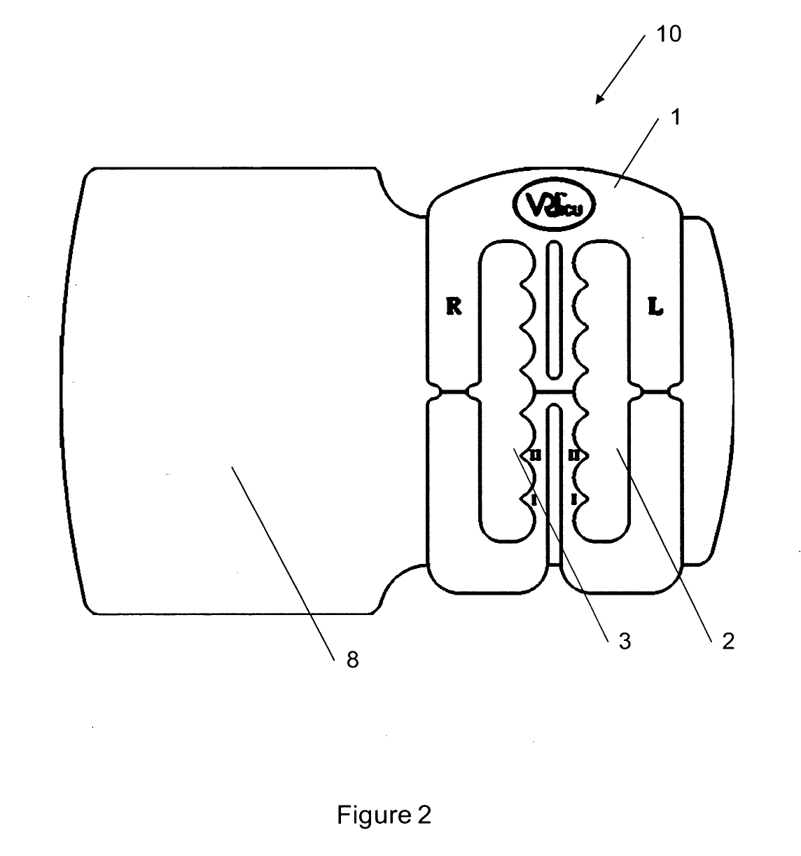

Reference is made to FIG. 2 illustrating more specifically a positioning unit 10 including a frame 1 defining two openings 2 and 3 configured to receive a pair of microphone matrices. The matrices are configured and operable to imagine the right and left sides of the patient. The corresponding right and left sides of the positioning unit 10 are marked respectively R and L. The openings 2 and 3 have geometry (shaped contour and size) defining an array of notches matching the geometry of the matrix-column to receive the microphones of said column within the notches, respectively. Therefore, one of the advantages of the positioning unit is to facilitate the correct placement of the matrices.

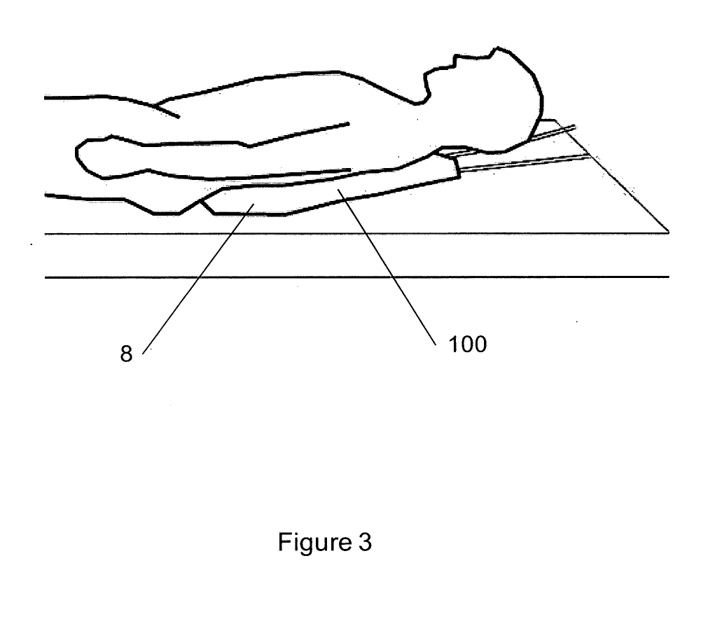

Reference is made to FIG. 3, illustrating the positioning of the diagnostic system of the present invention respectively to the subject to be examined. In use, the positioning unit is placed on a surface such as an examination table, a hospital bed or a patient bed, between the mattress and the patient. The matrix is aligned on the positioning unit and the patient lies on the positioning unit with the sensors positioned at a location of interest for the recording of acoustic signals. Alternatively, the recording procedure may be performed while the patient is sitting using vacuum vibration arrays.

When imaged, the patient lies in either a horizontal or slightly inclined ergonomic position with the patient's weight distributed on the patient's bed. This position is known to be more “ergonomic” and reduces the stress on the back of a patient and causes the blood flow to be less restricted than if the patient were sitting. This results in patients being more comfortable, which ultimately leads to less movement during the imaging process. It should be noted that reduction in patient movement during imaging may result in a higher throughput, a more consistent image quality which is free from motion artifacts. In addition, ergonomic positioning of a patient may aid in yielding images that allow for better prognosis or diagnosis.

In one aspect of the invention, the positioning unit 10 forms a disposable unit, with a disposable interface 8, such as a disposable latex-free cover that covers at least the entire upper surface, intended for use on a single patient only to prevent cross-contamination and avoid transmitting infection from one patient to the next. The interface is preferably formed from a hospital grade “clean-wrap”. The interface 8 may be a sheath configured and operable to cover the microphone array before laying the patient on the setup comprising the microphone array and the positioning unit.

In another aspect of the invention, the matrix can be aligned according to the patient's height. Turning back to FIG. 2 in this non-limiting example, the positioning unit 10 provides two height adjustable positions. The positioning unit 10 includes a frame 1 defining two openings 2 and 3 configured to receive a pair of microphone matrices. The openings 2 and 3 have geometry, i.e. shaped contour and size, defining an array of N spaced-apart notches matching the shaped contour of the matrix-column to receive separately M microphones of said column within the notches. The number of the notches (N) of the openings may be superior to the number of microphones of the matrix-column (M) enabling the displacement of the matrix along the openings, and several locations of the matrix within the frame. This configuration provides a degree of freedom in accurately positioning the patient on the microphones, enabling a plurality of height adjustable positions according to the patient stature. In this non-limiting example, two positioning markers are represented on the positioning unit 10, the positioning marker I is intended to patients smaller than or having a height of 180 cm, and the positioning marker II for patients taller than 180 cm. It should be noted that the matrix can be configured for individuals having smaller height or for children by reducing the number of rows.

According to another embodiment of the present invention, the matrix can also be positioned according to the patient's width. The positioning unit includes a frame defining two openings configured to receive a pair of microphone matrix. The openings have geometry, i.e. shaped contour and size, defining a 2D array of N notches matching the shaped contour of the matrix column and row to receive a 2D array of M microphones within the N notches, enabling several locations of the matrix within the frame. This configuration provides a degree of freedom in accurately positioning the patient on the microphones, enabling a plurality of width adjustable positions according to the patient width.

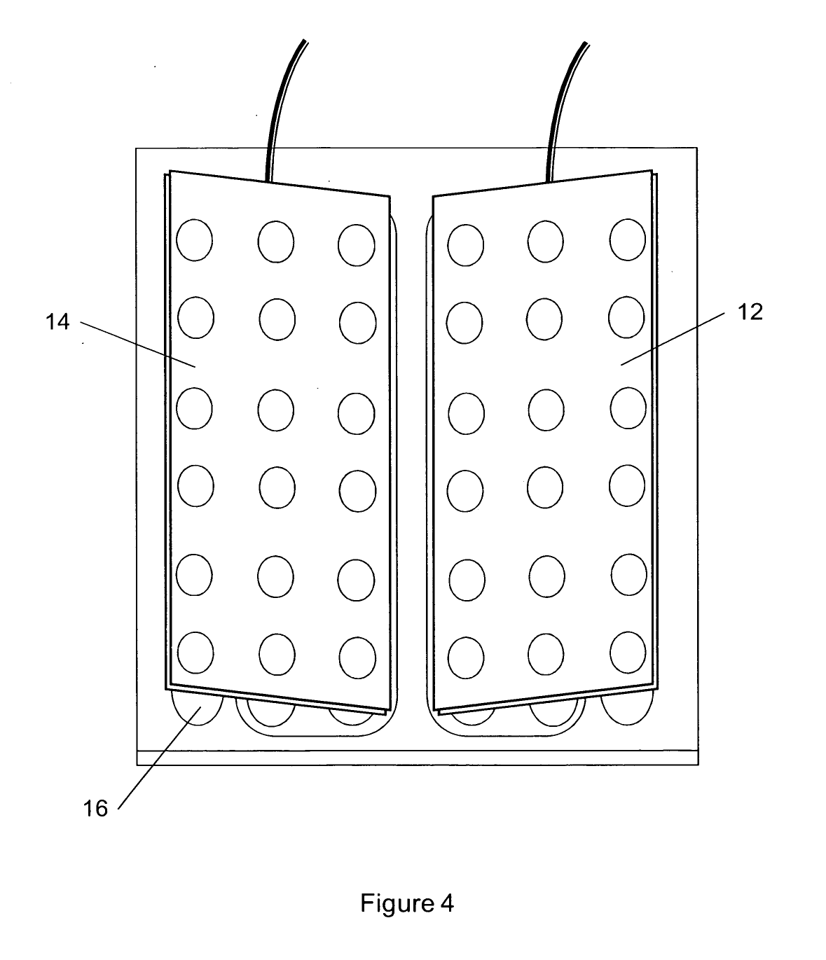

Reference is made to FIG. 4 illustrating a pair of ergonomic matrices (12 and 14) according to one embodiment of the present invention. The matrix is flexible and can conform to the shape of a surface applied to it. The pressure sensors may be encased in ergonomic capsules 16 made of latex-free material. When using the matrix, the pads that come into contact with the patient may be made from medical grade silicone and should cause no discomfort. For example, when the matrix is placed on the positioning unit and person's back is applied to the matrix, the matrix will conform to the shape of the person's back.

The ability to apply the matrix to a body region without the need for any fastening devices such as straps or vacuum, allows the matrix to be applied to a body over a prolonged period of time. The recording system may thus be used for continuously monitoring and recording acoustic signals over a substantially unlimited period of time. In particular, the system may be used to continuously monitor acoustic signals such as respiratory tract sounds or cardiac sounds in an individual in an intensive care unit.

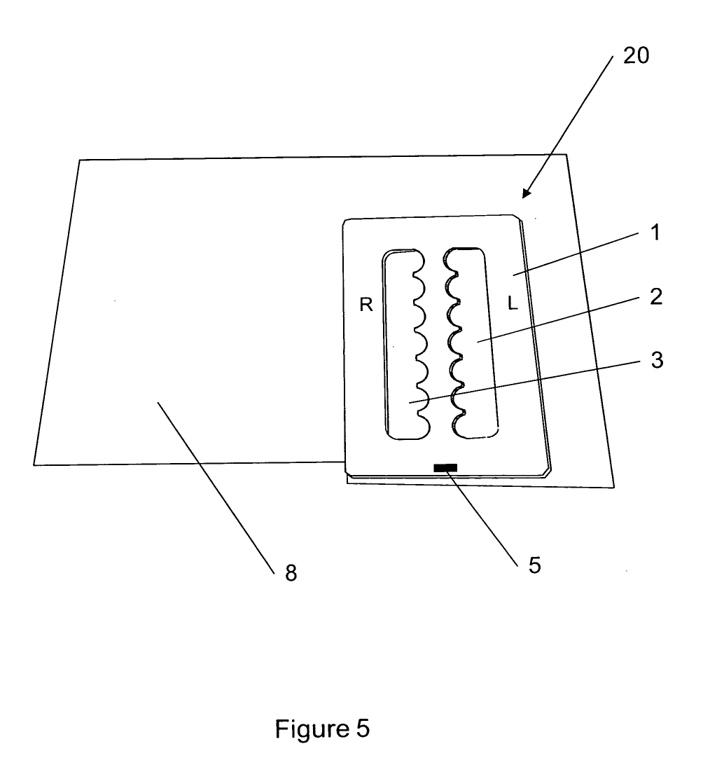

Reference is made to FIG. 5, illustrating another configuration of the positioning unit 20. A spine pointer 5 is marked on the positioning unit such that when the patient lays in either a horizontal or slightly inclined position on the positioning unit 20 with the spine pointer 5 pointing toward the spine of the patient, the patient's spine is properly aligned along the central area, and the side areas are proximate to the patient's sides.

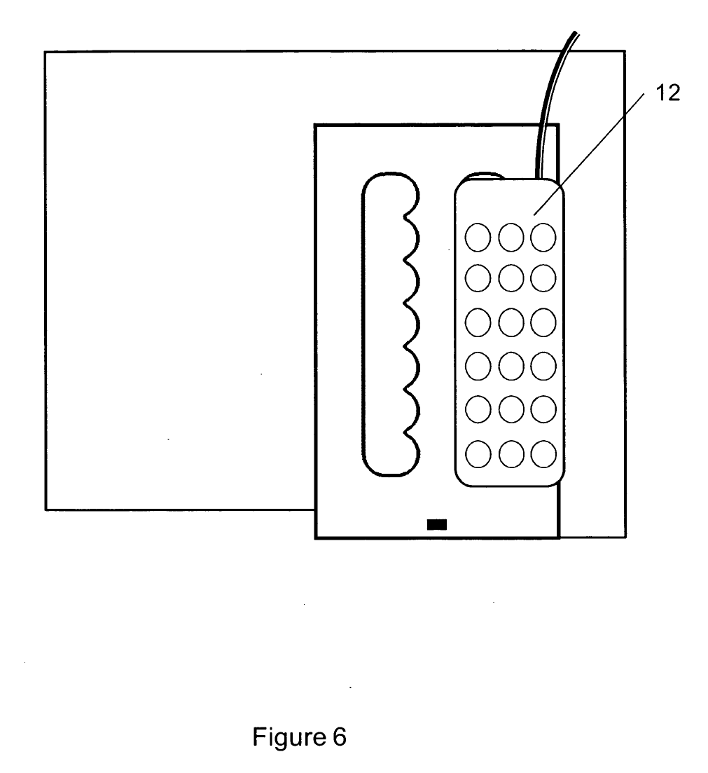

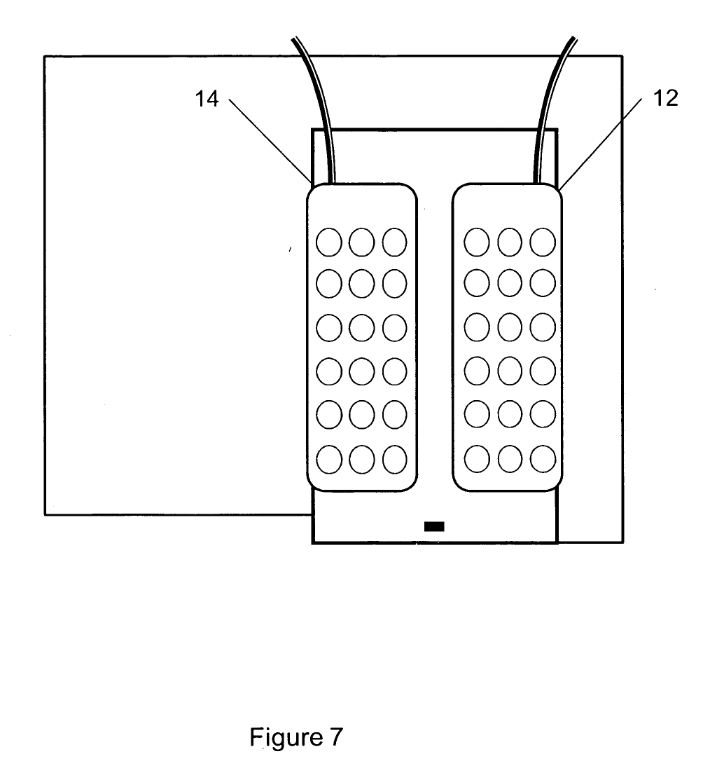

Reference is made to FIG. 6, illustrating the alignment of a left microphone array on the notched surface relief on the side of the positioning unit labeled L, laying it with the sensor pads facing upwards. FIG. 7 illustrates the alignment of the two microphone arrays on the notched surface reliefs on the sides of the positioning unit labeled L and R.

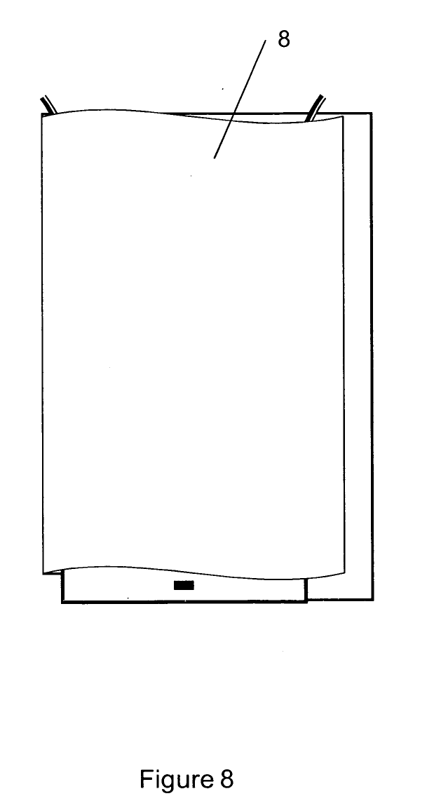

FIG. 8 illustrates the covering of the matrix with the sheath 8 attached to the positioning unit 10 by folding it over. The positioning unit is therefore configured and operable for the dual purpose of covering the matrices such that they do not come into direct contact with the patient's back, and to serve as a template for accurately positioning the left and right matrices in preparation for image acquisition.

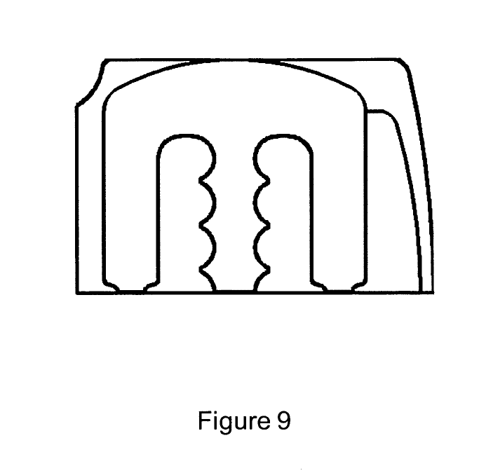

According to another embodiment, the positioning unit may be folded to facilitate the storage of such units. FIG. 9 illustrates the positioning unit in a folded shape.

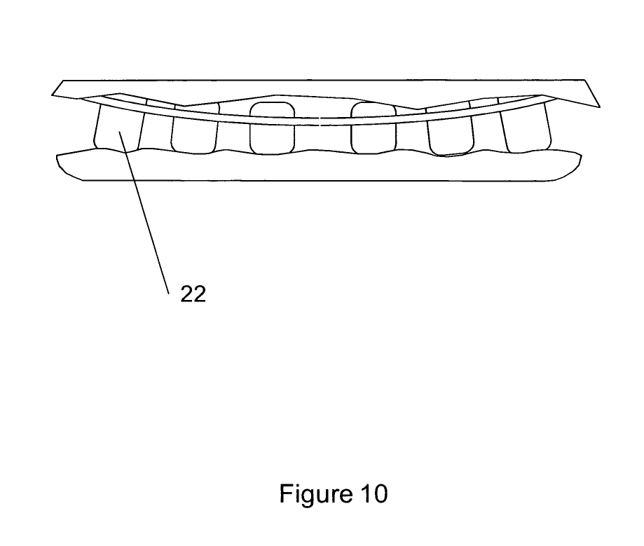

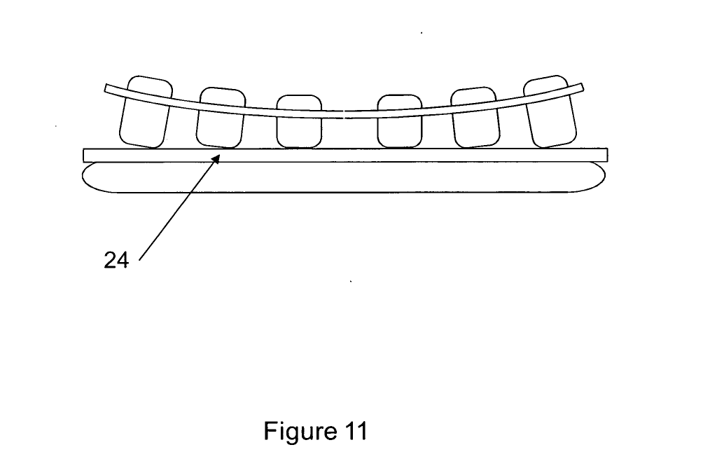

Reference is made now to FIG. 10-11 illustrating, the diagnostic system i.e. the positioning unit and the recording matrix attached thereto, configured as an inflating structure. The inflated structure is inflated to a pressure to effectively receive the body of the patient, enabling the full contact (i.e. the contact of all the sensors of the matrix) with the subject's body. As illustrated in FIG. 10, the recording matrix 22 may include an inflatable air chamber allowing the patient's body to be comfortably supported at an appropriate height to allow ergonomic alignment of the body to the positioning unit to facilitate comfortable positioning.

As illustrated in FIG. 11, the positioning unit 24 may also be inflated to facilitate the alignment of the recording matrix on the patient's bed.

1. A diagnostic system comprising at least one matrix carrying an array of sensors for recording signals from a body, and a positioning unit configured for alignment with said matrix, said positioning unit having a surface relief matching a surface relief of at least a part of said matrix, such that said positioning unit is adapted for engaging with at least part of the array of sensors matrix and maintaining said matrix when in operation, thereby enabling accurate positioning of the sensors with respect to a body region from which the signals are to be recorded.

2. The diagnostic system of claim 1, wherein said positioning unit comprises a frame made with at least one opening of a geometry defining said surface relief for accommodating at least one array of sensors.

3. The diagnostic system of claim 2, wherein said opening has a geometry matching the geometry of at least a region of the matrix.

4. The diagnostic system of claim 3, wherein said opening defines an array of notches matching the shaped contour of the matrix-column to receive the sensors of said column within the notches respectively.

5. The diagnostic system of claim 3, wherein said opening defines an array of N notches wherein N is superior to the number of the columns of the array of sensors matrix.

6. The diagnostic system of claim 5, configured to provide a plurality of height adjustable positions.

7. The diagnostic system of claim 5, configured to provide a plurality of width adjustable positions.

8. The diagnostic system of claim 1, wherein said positioning unit is disposable.

9. The diagnostic system of claim 1, configured and operable to ensure correct and ergonomic positioning.

10. The diagnostic system of claim 2, comprising two matrixes, said positioning unit having a frame made with two openings configured to accommodate said two matrices enabling the simultaneous recording of the acoustic signals of the right and the left side of a patient.

11. The diagnostic system of claim 1, wherein said positioning unit comprising a disposable interface covering at least the entire upper surface thereof.

12. The diagnostic system of claim 1, wherein a spine pointer is marked on said positioning unit such that the patient's spine is properly aligned relatively to the matrix.

13. The diagnostic system of claim 1, wherein said matrix is configured to be flexible and conform to the shape of a surface applied to it.

14. The diagnostic system of claim 1, wherein said positioning unit and/or said at least one matrix is/are configured as an inflating structure enabling the contact of all the sensors of the matrix with the patient's body.

15. A positioning unit for use in the diagnostic system of claim 1, said positioning unit being configured to define a surface relief matching a surface relief of at least a part of a multiple-sensor matrix, such that said positioning unit is adapted for engaging with the at least part of the matrix and maintaining said matrix when in operation.
